(19) United States
(12) Plant Patent Application Publication
(10) Pub. No.: US 2026/0206664 P1
(43) Pub. Date: Jul. 16, 2026

(54) LIME PLANT NAMED 'S 96-10'

(50) ;

(71) Applicant: Commonwealth Scientific and Industrial Research Organisation, Canberra ACT (AU)

(72) Inventor: Stephen Richard SYKES, Canberra ACT (AU)

(73) Assignee: Commonwealth Scientific and Industrial Research Organisation, Canberra ACT (AU)

(21) Appl. No.: 19/400,156

(22) Filed: Nov. 25, 2025

Related U.S. Application Data
(60) Provisional application No. 63/725,607, filed on Nov. 27, 2024.

(51) Int. Cl. A01H 6/78; A01H 5/08

(52) U.S. Cl. CPC A01H 6/785; A01H 5/08
USPC PLT/201

(57) ABSTRACT

The new lime plant variety ‘S 96-10’ is provided. The new plant variety is produced from a cross of ‘Nules Clementine’ (female parent, not patented) and ‘Australian Blood’ (male parent, not patented). The new plant variety produces fruit with rind that is dark burgundy colored. The internal flesh of the produced fruit of the new plant variety is bright pink to burgundy colored. A spreading growth habit is exhibited.

### CROSS REFERENCE TO RELATED APPLICATIONS

This application claims priority to U.S. Provisional Application No. 63/725,607, which was filed on Nov. 27, 2024, the contents of which are hereby incorporated by reference for all purposes.

Latin name of the genus and species: Genus and species—Clementina de Nules X (Citrus limonia Osbeck x Citrus australasica var. sanguinea).

Variety Denomination: The new lime plant claimed is of the variety denominated ‘S 96-10’.

### BACKGROUND OF THE INVENTION

The present invention relates to the discovery of a new and distinct cultivar of lime plant, referred to as ‘S 96-10’, as herein described and illustrated. The new plant variety has also been referred to as Breeder's code 94-02-10.

The new plant cultivar originated through controlled pollination in Victoria, Australia during 1994. Seeds were collected from mature fruit in 1995 and were sown. Seedlings were established in 1995, initially as potted plants, then planted in the field in Victoria, Australia. Selective study resulted in the identification of a single plant of the new variety. The new plant variety was identified for its fruit size, dark burgundy rind color, bright pink to burgundy color of its internal flesh, and ease of propagation.

The female parent of the new plant cultivar is ‘Nules Clementine’ (not patented). The male parent of the new plant cultivar is ‘Australian Blood’ (not patented). The Latin name of the genus and species of ‘Australian Blood’ has been referred to as Citrus limonia Osbeck x Citrus australasica var. sanguinea and alternatively as Citrus x sykesii Mabb.

‘S 96-10’ has been found to undergo asexual propagation by vegetative cuttings in Victoria, Australia. Asexual propagation techniques in Victoria, Australia, have shown that the characteristics of the new plant variety are homogenous, stable, and strictly transmissible by such asexual propagation from one generation to another. Accordingly, the new plant variety undergoes asexual propagation in a true-to-type manner with all the characteristics, as herein described, firmly fixed and retained through successive generations of such asexual reproduction.

### SUMMARY OF THE INVENTION

The following characteristics of the new plant cultivar ‘S 96-10’ have been repeatedly observed and are determined to be the unique characteristics of the new lime plant:
1) Dark burgundy rind color of fruit,
2) Bright pink to burgundy color of internal flesh of fruit, and
3) Fruit shape is pear.

### COMPARISON BETWEEN PARENTAL AND COMMERCIAL CULTIVARS

The new plant variety of the present invention can readily be distinguished from its ancestors. Moreover, the new plant variety can be readily distinguished from other similar non-parental varieties, for example, ‘Red Finger Limes’ (not patented).

TABLE 1
Comparison Table

| Denom- | | | State of |
|---|---|---|---|
| ination | Characteristic | State of | expression of |
| of similar | for | expression of | candidate variety |
| variety | comparison | similar variety | (‘S 96-10’) |
| ‘Nules | Fruit surface, | Orange | Dark burgundy |
| Clementine’ | predominant | | |
| | color | | |
| ‘Nules | Fruit width | Medium (6.47 ± | Small (4.22 ± |
| Clementine’ | (equatorial | 0.19 cm) | 0.33 cm) |
| | diameter) | | |
| ‘Nules | Fruit, presence | Absent | Present |
| Clementine’ | of neck | | |
| ‘Nules | Leaf blade | Long (9.46 ± | Medium (6.77 ± |
| Clementine’ | length | 0.48 cm) | 0.53 cm) |
| ‘Nules | Leaf blade | Medium (3.90 ± | Narrow (2.86 ± |
| Clementine’ | width | 0.57 cm) | 0.17 cm) |
| ‘Australian | Fruit surface, | Red to red | Dark burgundy |
| Blood’ | predominant | orange | |
| | color | to-burgundy | |
| ‘Australian | Fruit, diameter | Absent-Small | Medium |
| Blood’ | of core | | |
| ‘Australian | Fruit, presence | Absent | Present |
| Blood’ | of neck | | |
| ‘Australian | Fruit, presence | Absent | Present |
| Blood' | of depression | | |
| | at distal end | | |
| ‘Australian | Fruit, presence | Absent | Present |
| Blood’ | of constriction | | |
| | at stalk end | | |
| ‘Australian | Leaf blade, | Crenate | Sinuate |
| Blood’ | incisions of | | |
| | margin | | |
| ‘Red Finger | Fruit length | All longer | Shorter; dark |
| Limes’ | and/or skin | (>5 cm); | burgundy |
| | color at | red to dark | |
| | maturity | brown to | |
| | | maroon red | |

Values in brackets are mean ± standard deviation (n = 4 for fruit characteristics and n = 5 for vegetative characteristics).

### BRIEF DESCRIPTION OF THE DRAWINGS

The accompanying photographic illustrations show typical fruit specimens in full color of the new plant variety ‘S 96-10’. The colors are as nearly true as is reasonably possible in a color representation of this type. The color values cited in the detailed botanical description accurately describe the colors of the new lime.

FIG. 1—illustrates halved fruit of the new plant variety.

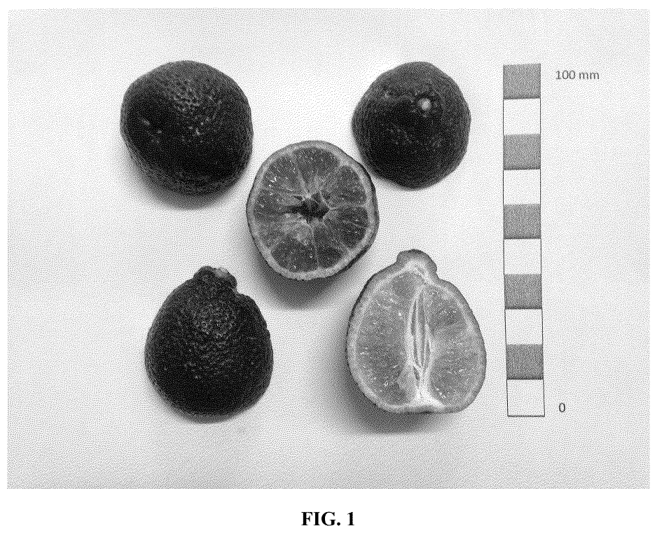

FIG. 2—illustrates a full fruit of the new plant variety-side view.

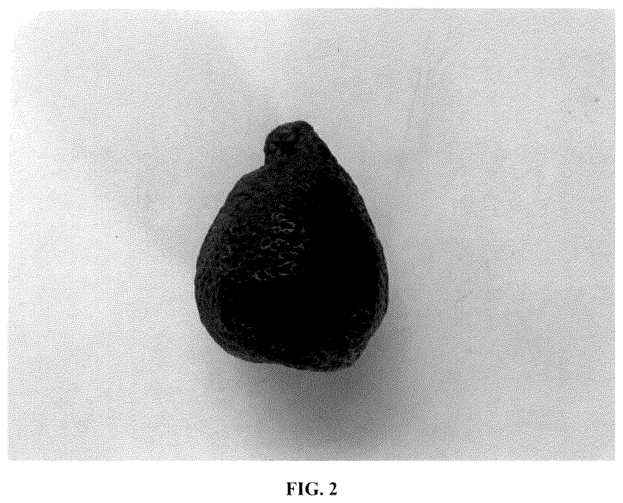

FIG. 3—illustrates specimens of fruit of the new plant variety-side view.

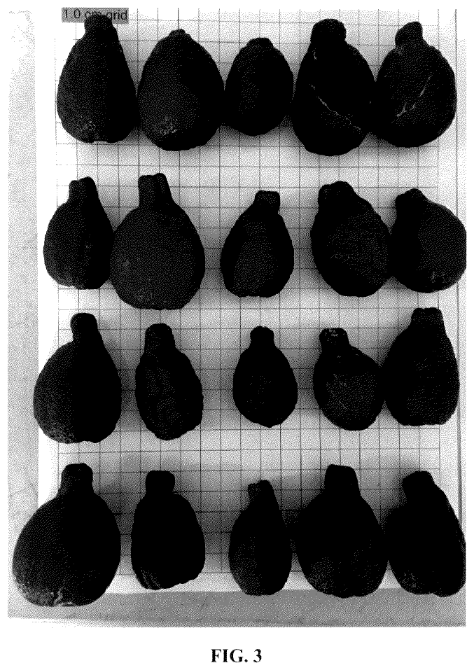

FIG. 4—illustrates specimens of fruit of the new plant variety (top of figure) compared to fruit of ‘Australian Blood’ (bottom of figure).

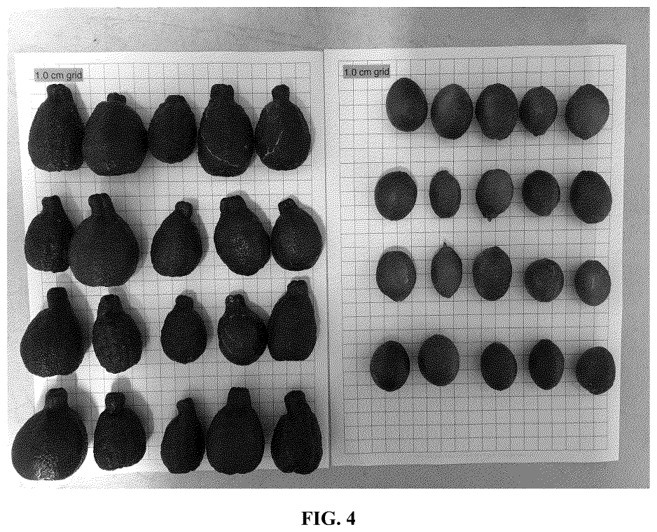

### DETAILED BOTANICAL DESCRIPTION

The following detailed description sets forth the distinctive characteristics of ‘S 96-10’. The data which defines these characteristics was collected from asexual reproductions of the original selection. Dimensions, sizes, colors, and other characteristics are approximations and averages set forth as accurately as possible. The color designations used herein represent the closest color observed on the majority of the specified botanical features. Where the color designations differ from the colors in the photographs, the color designations are accurate.

The new plant variety has not been observed under all possible environmental conditions. The phenotype may vary somewhat with variations in environment and cultural practices, such as temperature and light intensity, without any variance in genotype.

### GENERAL

Parentage:
   Female parent.—‘Nules Clementine’ (not patented).
   Male parent.—‘Australian Blood’ (not patented).

### PLANT

Ploidy.—Diploid.
Growth habit.—Spreading.
Tree height.—3.22 m.
Density of Spines.—Sparse.
Tree Length of Spines.—0.74 cm.
Self.—Incompatibility.—Absent.
Disease and Insect Resistance.—None observed to date.

### FOLIAGE

General:
   Young leaf.—Presence of anthocyanin coloration — Present.
   Young leaf.—Intensity of anthocyanin coloration — Very strong.
   Leaf blade.—Length — 67.70 mm.
   Leaf blade.—Width — 28.60 mm.
   Leaf blade.—Ratio length/width — 2.37.
   Leaf blade.—Shape in cross section — Weakly concave.
   Leaf blade.—Twisting — Absent.
   Leaf blade.—Green color — Medium.
   Leaf blade.—Undulation of margin — Weak.
   Leaf blade.—Incisions of margin — Crenate.
   Leaf blade.—Shape of apex — Acute.
   Leaf blade.—Margination at tip — Absent.
Petioles:
   Length.—7.27 mm.
   Presence of wings.—Present.
   Width of wings.—Very narrow.

### FLOWERS

General:
   Flower bud.—Presence of anthocyanin coloration — Present.
   Flower bud.—Intensity of anthocyanin coloration — Strong.
   Flowering habit.—Flowering once.
Calyx:
   Diameter.—25.9 mm.
Petal:
   Length.—14.3 mm.
   Width.—6.34 mm.
   Ratio length/width.—2.25.
Stamen:
   Length.—9.24 mm.
   Base union of stamens.—Present.
Anther:
   Viable pollen.—Present.
   Color.—Medium yellow.
Style:
   Length.—6.05 mm.

### FRUIT

General:
   Time of maturity of fruit for consumption.—Early to medium; 60 days, mid-May to mid-July.
   Clustering of fruits.—Absent.
   Fruit.—Parthenocarpy — Parthenocarpic tendency identified.
   Fruit.—Shape — Periform.
   Fruit.—Length — 5.57 cm.
   Fruit.—Diameter — 4.22 cm.
   Fruit.—Ratio length/diameter — 1.32.
   Fruit.—Height — 55.71 mm.
   Fruit.—Width — 42.21 mm.
   Fruit.—Weight — 47.30 g.
   Fruit.—Position of broadest part — Towards distal end.
   Fruit.—General shape of proximal part — Slightly rounded.
   Fruit.—Presence of neck — Present.
   Fruit.—Length of neck — 9.96 mm.
   Fruit.—General shape of distal part — Flattened.
   Fruit.—Presence of nipple — Absent.
   Fruit.—Diameter of stylar scar — 1.16 mm.
   Fruit.—Persistence of style — None.
   Fruit.—Presence of radial grooves at distal end — Present.
   Fruit.—Expression of radial grooves at distal end — Strong.
   Fruit.—Pedicel length: 7.27 mm.
   Fruit.—Color variegation — Absent.
   Fruit surface.—Color — Deep burgundy.
   Fruit surface.—Glossiness — Medium.
   Fruit surface.—Roughness — Medium.
   Fruit surface.—Size of oil glands — All more or less the same size.
   Fruit surface.—Size of larger oil glands — Small.
   Fruit surface.—Conspicuousness of larger oil glands — Medium.
   Fruit surface.—Presence of pitting and pebbling on oil glands — Pitting present, pebbling absent.
   Fruit surface.—Density of pitting — Medium.
   Fruit rind.—Thickness — 2.33 mm.
   Fruit rind.—Oiliness — Medium.
   Fruit.—Main color of flesh — Medium pink to burgundy.
   Fruit.—Filling of core — Absent.
   Fruit.—Diameter of core — 7.09 mm.
   Fruit.—Presence of rudimentary segments — Absent.
   Fruit.—Number of well developed segments — 8.38.
   Fruit.—Strength of segment walls — Strong.
   Fruit.—Length of juice vesicles — Medium to long.
   Fruit.—Thickness of juice vesicles — Medium.
   Fruit.—Conspicuousness of juice vesicle walls — Medium.
   Fruit.—Coherence of juice vesicles — Medium to strong.
   Fruit.—Juiciness — High.
   Fruit juice.—Total soluble solids — 10.22%.
   Fruit juice.—Acidity — pH of 3.13
   Fruit.—Strength of fibre — Strong.

### SEED

Seed abundance in fruit.—Few to medium; 4.90.
Polyembryony.—Absent.

1. A new and distinct variety of lime plant named ‘S 96-10’, substantially as illustrated and described herein.
